(19) United States
(12) Patent Application Publication
(10) Pub. No.: US 2024/0042350 A1
(43) Pub. Date: Feb. 8, 2024

(54) FILTER

(71) Applicant: Murata Manufacturing Co., Ltd., Nagaokakyo-shi (JP)

(72) Inventors: Masaru BANJU, Nagaokakyo-shi (JP); Takashi KONDO, Nagaokakyo-shi (JP); Shusuke YOKOTA, Nagaokakyo-shi (JP)

(21) Appl. No.: 18/490,955

(22) Filed: Oct. 20, 2023

Related U.S. Application Data
(63) Continuation of application No. PCT/JP22/24001, filed on Jun. 15, 2022.

(30) Foreign Application Priority Data
Jul. 7, 2021 (JP) 2021-112812

(51) Int. Cl. B01D 29/07; B01D 35/30; B01D 39/10

(52) U.S. Cl. CPC B01D 29/07; B01D 35/306; B01D 39/10; B01D 2201/0415

(57) ABSTRACT

A filter that includes: a filtering portion having a first main surface, a second main surface opposite to the first main surface, and a plurality of through holes connecting the first main surface and the second main surface with each other, wherein the filtering portion includes one or more curved portions warped in a direction toward a side of the first main surface or a side of the second main surface.

### CROSS REFERENCE TO RELATED APPLICATIONS

The present application is a continuation of International application No. PCT/JP2022/024001, filed Jun. 15, 2022, which claims priority to Japanese Patent Application No. 2021-112812, filed Jul. 7, 2021, the entire contents of each of which are incorporated herein by reference.

### TECHNICAL FIELD

The present disclosure relates to a filter.

### BACKGROUND ART

Examples of filters for capturing cells include a cell-capture metal filter disclosed in Patent Document 1.
Patent Document 1: Japanese Unexamined Patent Application Publication No. 2015-188323

### SUMMARY OF THE INVENTION

However, the filter disclosed in Patent Document 1 still has room to be improved regarding usability.

The present disclosure provides a filter with high usability.

A filter according to an aspect of the present disclosure includes: a filtering portion having a first main surface, a second main surface opposite to the first main surface, and a plurality of through holes connecting the first main surface and the second main surface with each other, wherein the filtering portion includes one or more curved portions warped in a direction toward a side of the first main surface or a side of the second main surface.

The present disclosure provides a filter with high usability.

### BRIEF DESCRIPTION OF THE DRAWINGS

FIG. 1 is a perspective view of an example of a filter according to Embodiment 1 of the present disclosure from a first main surface side.

FIG. 2 is a perspective view of the example of the filter according to Embodiment 1 of the present disclosure from a second main surface side.

FIG. 3 is a front view of the example of the filter according to Embodiment 1 of the present disclosure.

FIG. 4 is a rear view of the example of the filter according to Embodiment 1 of the present disclosure.

FIG. 5 is a left side view of the example of the filter according to Embodiment 1 of the present disclosure.

FIG. 6 is a right side view of the example of the filter according to Embodiment 1 of the present disclosure.

FIG. 7 is a plan view of the example of the filter according to Embodiment 1 of the present disclosure.

FIG. 8 is a bottom view of the example of the filter according to Embodiment 1 of the present disclosure.

FIG. 9 is an enlarged view of part of a filtering portion.

FIG. 10 is an enlarged view of part of a reinforcement in the filtering portion.

FIG. 11 is a cross-sectional view of the filtering portion in FIG. 10 taken along line A-A.

FIG. 12 is an enlarged cross-sectional view of part of a curved portion.

FIG. 13 is an enlarged perspective view of part of a curved portion.

FIG. 14A is a schematic diagram illustrating an example of a method of manufacturing a filter.

FIG. 14B is a schematic diagram illustrating the example of the method of manufacturing a filter.

FIG. 14C is a schematic diagram illustrating the example of the method of manufacturing a filter.

FIG. 14D is a schematic diagram illustrating the example of the method of manufacturing a filter.

FIG. 14E is a schematic diagram illustrating the example of the method of manufacturing a filter.

FIG. 14F is a schematic diagram illustrating the example of the method of manufacturing a filter.

FIG. 14G is a schematic diagram illustrating the example of the method of manufacturing a filter.

FIG. 14H is a schematic diagram illustrating the example of the method of manufacturing a filter.

FIG. 15 is a table illustrating the results of an experiment in which the amount of warpage is measured with current density taken as a parameter.

FIG. 16 is a perspective view of a filter of a variation from a first main surface side.

FIG. 17 is a perspective view of the filter of the variation from a second main surface side.

FIG. 18 is a front view of the filter of the variation.

FIG. 19 is a rear view of the filter of the variation.

FIG. 20 is a left side view of the filter of the variation.

FIG. 21 is a right side view of the filter of the variation.

FIG. 22 is a plan view of the filter of the variation.

FIG. 23 is a bottom view of the filter of the variation.

### DETAILED DESCRIPTION OF THE PREFERRED EMBODIMENTS

(Background Leading to Present Disclosure)

The filter described in Patent Document 1 is flat and plate-shaped. Hence, the filter does not have a portion to hold, and it is difficult to handle the filter in some cases.

For example, when a user removes the filter from a holder after filtration, the user uses tweezers or the like to pinch an outer edge portion of the filter to pick it up. However, a flat plate-shaped filter has a problem in that it is not easy to insert the tips of tweezers on both sides of an outer edge portion of a flat plate-shaped filter, and hence it is difficult to pick up. In addition, when the user uses tweezers to pinch the filter to picks it up, the tips of the tweezers come into contact with an outer edge portion of the filter. A force acting on the outer edge portion of the filter can break the filter.

The flat plate-shaped filter also has a problem in that it is difficult to distinguish between the front and back surfaces.

To solve these problems, the inventors have conceived a configuration of a warped filter, leading to the invention described below.

A filter according to an aspect of the present disclosure includes: a filtering portion having a first main surface, a second main surface opposite to the first main surface, and a plurality of through holes connecting the first main surface and the second main surface with each other, wherein the filtering portion includes one or more curved portions warped in a direction toward a side of the first main surface or a side of the second main surface.

The above configuration improves the usability.

The filter may further include a frame surrounding a periphery of the filtering portion and extending along an outer peripheral shape of the filtering portion.

This configuration makes it easy to form the curved portion while improving the mechanical strength of the filter.

The filter may further include a reinforcement on the filtering portion and being thicker than the filtering portion.

This configuration improves the mechanical strength of the filter.

The reinforcement may include a plurality of first reinforcement members extending in a first direction and a plurality of second reinforcement members extending in a second direction intersecting the first direction when viewed from the side of the first main surface, and the one or more curved portions may be warped to the side of the first main surface or the side of the second main surface in a third direction intersecting the first direction and the second direction when viewed from the side of the first main surface.

This configuration makes it possible to control the direction in which the curved portion is warped by utilizing the shape of the reinforcement.

An amount of warpage of the one or more curved portions may be 4×10⁻⁴ times an outer diameter of the filter to 0.1 times the outer diameter of the filter.

This configuration further improves the usability.

The filter base may have a flat portion where the first main surface and the second main surface are flat, the flat portion being located in a center of the filter base portion, and the one or more curved portions include first and second curved portions positioned such that the flat portion is located in between the first and second curved portions in a cross-sectional view of the filter.

This configuration further improves the usability.

A ratio of an area occupied by the one or more curved portions in the first main surface may be 1% to 100%.

This configuration further improves the usability.

The filter may contain at least one of a metal and a metal oxide as a main component thereof.

This configuration further improves the usability while improving the mechanical strength.

Hereinafter, Embodiment 1 of the present disclosure will be described with reference to the accompanying drawings. In each figure, each component is exaggerated for convenience of explanation.

### Embodiment 1

[Overall Configuration]

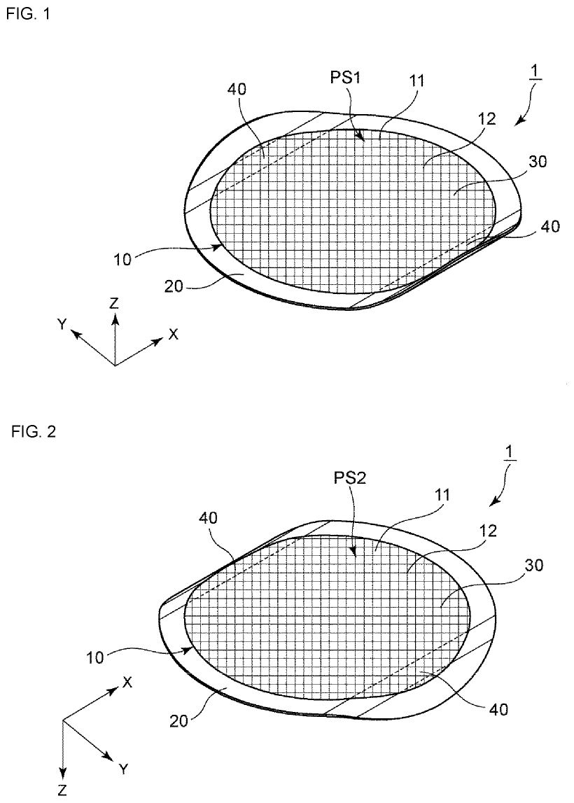

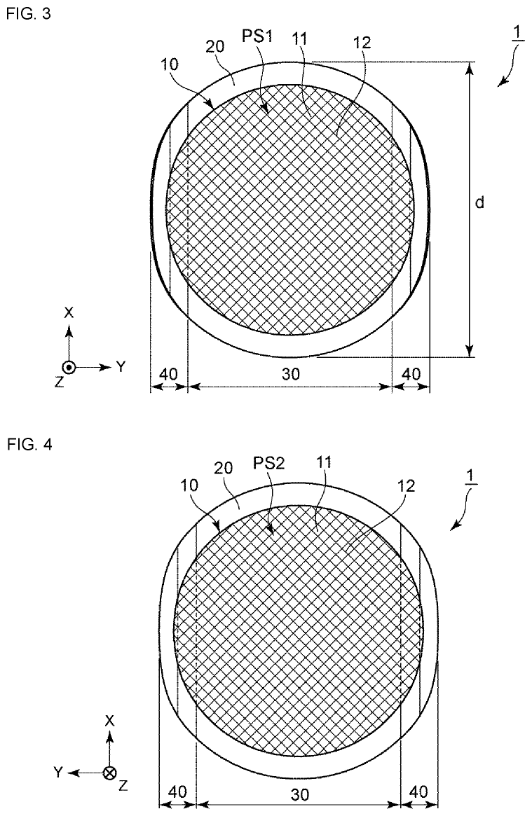

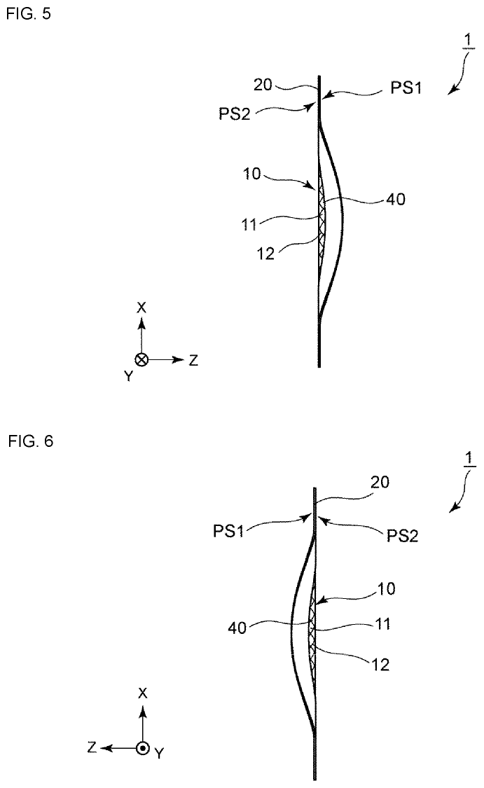

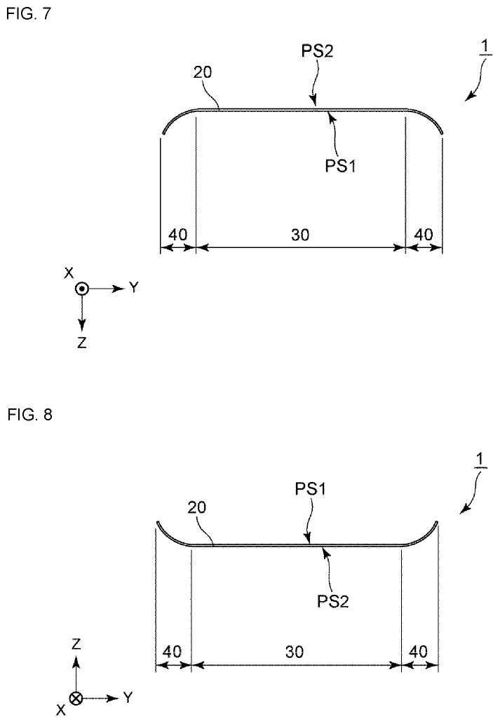

FIG. 1 is a perspective view of an example of a filter 1 according to Embodiment 1 of the present disclosure from a first main surface PS1 side. FIG. 2 is a perspective view of the example of the filter 1 according to Embodiment 1 of the present disclosure from a second main surface PS2 side. FIG. 3 is a front view of the example of the filter 1 according to Embodiment 1 of the present disclosure. FIG. 4 is a rear view of the example of the filter 1 according to Embodiment 1 of the present disclosure. FIG. 5 is a left side view of the example of the filter 1 according to Embodiment 1 of the present disclosure. FIG. 6 is a right side view of the example of the filter 1 according to Embodiment 1 of the present disclosure. FIG. 7 is a plan view of the example of the filter 1 according to Embodiment 1 of the present disclosure. FIG. 8 is a bottom view of the example of the filter 1 according to Embodiment 1 of the present disclosure. In the figures, the X, Y, and Z directions correspond to the longitudinal direction, the lateral direction, and the thickness direction of the filter 1, respectively.

For example, the filter 1 is a filter configured to filter a fluid containing a filtration target substance.

In this specification, the filtration target substance denotes a target substance to be filtered out among substances contained in a fluid. For example, a filtration target substance may be a biological substance contained in a fluid. The biological substance denotes a substance derived from living things such as cells (eucaryotes), bacteria (true bacteria), and viruses. Examples of cells (eucaryotes) include induced pluripotent stem cells (iPS cells), ES cells, stem cells, mesenchymal stem cells, mononuclear cells, single cells, cells clusters, planktonic cells, adherent cells, nerve cells, white blood cells, regenerative medicine cells, autologous cells, cancer cells, circulating tumor cells (CTC), HL-60, HELA, and fungi. Examples of bacteria (true bacteria) include colon bacilli and tubercle bacilli.

Examples of fluid include liquid and gas. Examples of liquid include cell suspensions.

The filter 1 is a metal filter. The material of the filter 1 contains at least one of a metal and a metal oxide as a main component. The material of the filter 1 may be, for example, gold, silver, copper, platinum, nickel, palladium, titanium, an alloy of any of these, and an oxide of any of these. In particular, the use of titanium or a nickel-palladium alloy reduces the amount of metal elution and the influence on the filtration target substance.

As illustrated in FIGS. 1 to 8, the filter 1 includes a filtering portion 10 and a frame 20 located in the outer periphery of the filtering portion 10. The filter 1 has the first main surface PS1 and the second main surface PS2 opposite to the first main surface PS1. In Embodiment 1, the filtering portion 10 and the frame 20 are integrally formed. The first main surface PS1 and the second main surface PS2 are opposed to each other.

<Filtering Portion>

The filtering portion 10 is configured to filter a fluid containing a filtration target substance. The filtering portion 10 includes a filter base 12 having a plurality of through holes 11 causing the first main surface PS1 and the second main surface PS2 to communicate with each other.

The filtering portion 10 includes a flat portion 30 where the first main surface PS1 and the second main surface PS2 are flat and a plurality of curved portions 40 formed with the flat portion 30 in between.

The flat portion 30 is located in the center of the filtering portion 10. Specifically, when viewed from the first main surface PS1 side, the flat portion 30 extends from the center of the filtering portion 10 in the X direction to outer peripheral portions of the filter 1.

In cross-sectional view, the plurality of curved portions 40 are located at parts of the outer periphery of the filter 1 with the flat portion 30 in between. The plurality of curved portions 40 are warped to the first main surface PS1 side. Specifically, the plurality of curved portions 40 are curved in the direction from the second main surface PS2 toward the first main surface PS1. The plurality of curved portions 40 have curved shapes in a free state in which no external force is acting on the filter 1. In Embodiment 1, the filtering portion 10 has two curved portions 40 opposed to each other in the Y direction. Thus, as illustrated in FIGS. 7 and 8, a cross section of the filter 1 cut in the Y direction is approximately U-shaped.

The shape of the filtering portion 10 is, for example, circular, polygonal, or elliptical when viewed in the thickness direction of the filter 1 (the Z direction). In Embodiment 1, the shape of the filtering portion 10 is approximately circular. Note that in this specification, “approximately circular” denotes that the ratio of the length of the major axis to the length of the minor axis is 1.0 to 1.2.

<Frame>

The frame 20 is located in the outer periphery of the filtering portion 10, and in the frame 20, the number of through holes 11 per unit area is smaller than in the filtering portion 10. The number of through holes 11 in the frame 20 is 1% or less of the number of through holes 11 in the filtering portion 10. The frame 20 may be thicker than the filtering portion 10. This configuration increases the mechanical strength of the filter 1.

The frame 20 surrounds the outer periphery of the filtering portion 10 and has a shape along the outer peripheral shape of the filtering portion 10. The frame 20 includes parts of the flat portion 30 and parts of the plurality of curved portions 40 in the outer peripheral portions of the filter 1. Specifically, the portions of the frame 20 located in the flat portion 30 of the filtering portion 10 are flat. The portions of the frame 20 located in the curved portions 40 of the filtering portion 10 are curved along the shape of the curved portion 40. Specifically, the portions of the frame 20 located in the curved portions 40 are warped to the first main surface PS1 side.

When the filter 1 is connected to a device and used, the frame 20 may function as a connecting portion for connecting the filter 1 to the device. In addition, the frame 20 may display information on the filter 1 (for example, dimensions of the through holes 11).

The frame 20 has a ring shape when viewed from the first main surface PS1 side of the filtering portion 10. The center of the frame 20 is aligned with the center of the filtering portion 10 when viewed from the first main surface PS1 side of the filter 1. In other words, the frame 20 has a circular shape concentric with the filter 1.

The following describes the filtering portion 10 in detail.

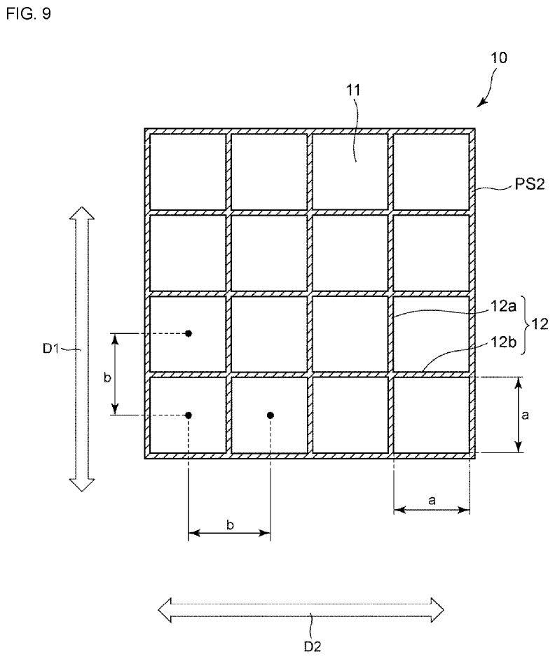

FIG. 9 is an enlarged view of part of the filtering portion 10. As illustrated in FIG. 9, the plurality of through holes 11 are regularly spaced on the first main surface PS1 and the second main surface PS2 of the filtering portion 10. Specifically, the plurality of through holes 11 are arranged at equal intervals in a matrix in the filtering portion 10.

In Embodiment 1, the plurality of through holes 11 are arranged in two arrangement directions parallel to each side of squares when viewed from the second main surface PS2 side of the filtering portion 10 (in the Z direction). As described above, a square lattice arrangement of the plurality of through holes 11 increases the ratio of openings and reduces the resistance of the filter 1 to fluid. This configuration shortens the process time and reduces stress on the filtration target substance. In addition, since the degree of the symmetry of the arrangement of the plurality of through holes 11 is high, it is easy to observe the filter 1.

Note that the arrangement of the plurality of through holes 11 is not limited to a square lattice arrangement and may be, for example, a quasi-periodic array or a periodic array. Examples of period arrays include an oblong array in which the intervals in the two arrangement directions are not equal as an example of a rectangular array and also include a triangular lattice array and a regular triangular lattice arrays. Note that a plurality of through holes 11 need only to be formed in the filtering portion 10, and the arrangement is not particularly limited.

The portion of the filtering portion 10 where the through holes 11 are not present is the filter base 12. As illustrated in FIG. 9, the filter base 12 is formed in a lattice shape. Specifically, the filter base 12 includes, in the filtering portion 10, a plurality of first base members 12a extending in a first direction D1 and a plurality of second base members 12b extending in a second direction D2 intersecting the first direction D1. The first direction D1 and the second direction D2 intersect each other on the XY plane. The plurality of first base members 12a are arranged at equal intervals in the second direction D2. The plurality of second base members 12b are arranged at equal intervals in the first direction D1.

Each of the plurality of first base members 12a and the plurality of second base members 12b is plate-shaped. The plurality of first base members 12a and the plurality of second base members 12b intersecting one another define the plurality of through holes 11. In Embodiment 1, when the filtering portion 10 is viewed from the second main surface PS2 side, the first direction D1 and the second direction D2 are orthogonal to each other.

In Embodiment 1, the plurality of first base members 12a and the plurality of second base members 12b are integrally formed.

The thickness of the filter base 12 in the filtering portion 10 is 0.5 μm to 20 μm. This configuration makes it possible to achieve a sufficient mechanical strength and reduces the pressure loss of the fluid passing through the filter. It is preferable that the thickness of the filter base 12 in the filtering portion 10 be 1.0 μm to 3 μm. This configuration further reduces the pressure loss of the fluid passing through the filter 1.

In Embodiment 1, the thickness of the filter base 12 is designed to be approximately uniform. An approximately uniform thickness of the filter base 12 enables good reproducibility in controlling the positions of the curved portions 40 and the amount of warpage. The term “approximately uniform” denotes that errors in the thickness of the filter base 12 are within a range of ±5%. Note that the thickness of the filter base 12 is not limited to being approximately uniform.

The interval b of the through holes 11 is designed as appropriate in accordance with the filtration target substance to be isolated. For example, in the case in which the filtration target substance is cells, the interval b of the through holes 11 is designed as appropriate in accordance with the type of cell (size, shape, characteristics, and elasticity) and the amount. Here, as illustrated in FIG. 9, the interval b of the through holes 11 denotes the distance between the center of a through hole 11 and the center of an adjacent through hole 11 when the through holes 11 are viewed from the second main surface PS2 side of the filtering portion 10. In Embodiment 1, the through hole 11 is a square when viewed from the second main surface PS2 side. The center of the through hole 11 is the intersection point of the two diagonal lines.

In the case of a periodic-array structure, the interval b of the through holes 11 is, for example, larger than the length a of one side of the through hole 11 and smaller than or equal to 10 times the length a, and preferably smaller than or equal to three times the length a of one side of the through hole 11. Alternatively, for example, the opening ratio of the filtering portion 10 is 10% or more, and preferably 25% or more. This configuration reduces the resistance of the filtering portion 10 to the fluid. Thus, this configuration shortens the process time and reduces stress on cells. Note that the opening ratio is calculated by (the area occupied by the through holes 11)/(the projected area of the second main surface PS2 on the assumption of no through hole 11 in the second main surface PS2).

Through the wall surface of the through hole 11, the opening in the first main surface PS1 and the opening in the second main surface PS2 communicate with each other. Specifically, the through hole 11 is configured such that the opening in the first main surface PS1 can be projected to the opening in the second main surface PS2. In other words, the through hole 11 is configured such that the opening in the second main surface PS2 overlaps the opening in the first main surface PS1 when the filtering portion 10 is viewed from the second main surface PS2 side. In Embodiment 1, the inner walls defining each through hole 11 are perpendicular to the first main surface PS1 and the second main surface PS2.

The shape of each through hole 11 is a square when viewed from the second main surface PS2 side, and the length a of one side of the through hole 11 is 0.5 μm to 400 μm. It is preferable that the length a of one side of the through hole 11 be 1 μm to 30 μm.

Note that the shape of the through hole 11 is not limited to a square when viewed from the second main surface PS2 side. For example, the shape of the through hole 11 when viewed from the second main surface PS2 side may be circular, elliptical, rectangular, polygonal, or the like.

The surface roughness of the first main surface PS1 and the second main surface PS2 in the filtering portion 10 should preferably be lower. Here, the surface roughness is a value measured by profiling a surface by using a stylus profiler at five points, calculating the difference between the maximum value and the minimum value at each point, and calculating the average value of the five differences. In Embodiment 1, the surface roughness should preferably be smaller than the size of the filtration target substance, or more preferably be smaller than half the size of the filtration target substance. This is because the adhesion of the filtration target substance can be reduced, and the filtration target substance can be collected with high efficiency after being caught by the filter 1.

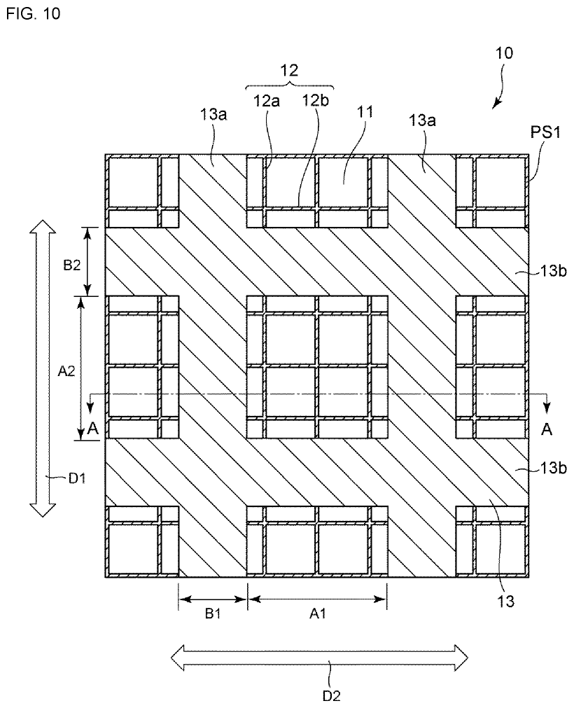

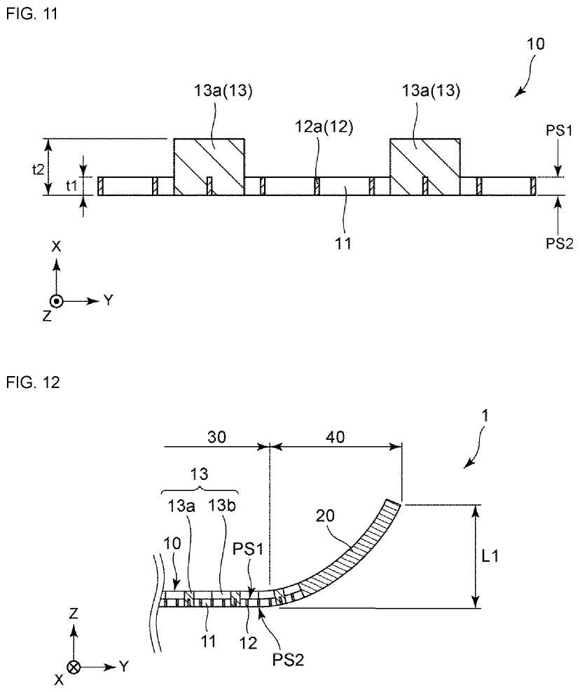

FIG. 10 is an enlarged view of part of a reinforcement 13 in the filtering portion 10. FIG. 11 is a cross-sectional view of the filtering portion 10 in FIG. 10 taken along line A-A.

As illustrated in FIGS. 10 and 11, the filtering portion 10 has the reinforcement 13. The reinforcement 13, which is a member to reinforce the filter base 12, improves the mechanical strength of the filter 1. For example, when a fluid containing a filtration target substance passes through the filtering portion 10, and an external force acts on the filter base 12, the reinforcement 13 prevents the filter base 12 from breaking.

The reinforcement 13 is provided on the first main surface PS1 of the filter base 12. The reinforcement 13 has a thickness t2 greater than the thickness t1 of the filter base 12.

The reinforcement 13 has a lattice shape when viewed from the first main surface PS1 side. The reinforcement 13 includes a plurality of first reinforcement members 13a extending in the first direction D1 and a plurality of second reinforcement members 13b extending in the second direction D2 intersecting the first direction D1. In Embodiment 1, the plurality of first reinforcement members 13a and the plurality of second reinforcement members 13b are orthogonal to one another.

Each of the plurality of first reinforcement members 13a and the plurality of second reinforcement members 13b is plate-shaped. The plurality of first reinforcement members 13a and the plurality of second reinforcement members 13b are integrally formed. The plurality of first reinforcement members 13a and the plurality of second reinforcement members 13b are located on the filter base 12 such that a plurality of through holes 11 are between adjacent reinforcement members.

The plurality of first reinforcement members 13a and the plurality of second reinforcement members 13b are arranged at equal intervals. For example, the interval A1 of the first reinforcement members 13a and the interval A2 of the second reinforcement members 13b are 200 μm to 500 μm. It is preferable that the intervals A1 and A2 be 250 μm to 350 μm. Note that the interval A1 denotes the distance between two adjacent first reinforcement members 13a. The interval A2 denotes the distance between two adjacent second reinforcement members 13b. In Embodiment 1, the interval A1 and the interval A2 are approximately equal. Note that the interval A1 and the interval A2 may be different.

The width B1 of each of the first reinforcement members 13a and the width B2 of each of the second reinforcement members 13b are greater than the width of each of the first base members 12a and the second base members 12b of the filter base 12, when the filtering portion 10 is viewed from the first main surface PS1 side. For example, the width B1 of each of the first reinforcement members 13a and the width B2 of each of the second reinforcement members 13b are 5 μm to 40 μm. It is preferable that the width B1 and the width B2 be 10 μm to 30 μm.

Next, the curved portions 40 will be described in detail.

FIG. 12 is an enlarged cross-sectional view of part of a curved portion 40. As illustrated in FIG. 12, the curved portion 40 consists of an outer peripheral end portion of the filter 1 warped so as to be curled up to the first main surface PS1 side. Specifically, as the position approaches the outer periphery of the filter 1, the curved portion 40 is curved continuously to the first main surface PS1 side. The term “curved continuously” denotes that it is curved gently without a step. In Embodiment 1, the curved portion 40 is curved in an arch shape.

The amount of warpage L1 of the curved portion 40 is 4×10⁻⁴ times the outer diameter d of the filter to 0.1 times the outer diameter d of the filter. It is preferable that the amount of warpage L1 of the curved portion 40 be 4×10⁻³ times the outer diameter d of the filter to 0.1 times the outer diameter d of the filter. It is more preferable that the amount of warpage L1 of the curved portion 40 be 0.02 times the outer diameter d of the filter to 0.1 times the outer diameter d of the filter. Note that the amount of warpage L1 denotes the distance from the plane including the second main surface PS2 of the flat portion 30 to the portion of the curved portion 40 at which the warpage is largest, in other words, the portion farthest away from the plane including the second main surface PS2 in the thickness direction of the filter 1 (the Z direction). In Embodiment 1, outer peripheral end portions of the frame 20 correspond to the largest warpage portions of the curved portion 40. For example, the amount of warpage L1 can be measured by placing the filter 1 with the second main surface PS2 in contact with a flat surface and measuring the distance between the flat surface and an outer peripheral end portion of the frame 20 in the Z direction.

For example, the amount of warpage L1 of the curved portion 40 needs to be 10 μm to 2.5 mm. This configuration makes it easy for a tool such as tweezers having processed thin tips to hold the curved portion 40. Alternatively, blowing (with an air blow gun) compressed gas makes it possible to increase the bend of the curved portion 40 and make a chance to hold it. It is preferable that the amount of warpage L1 of the curved portion 40 be 100 μm to 2.5 mm. This configuration prevents the filter 1 from being damaged when the filter 1 is held with a tool such as commercially available tweezers. It is more preferable that the amount of warpage L1 of the curved portion 40 be 0.5 mm to 2.5 mm. With this configuration, the shapes of the curved portions 40 can be maintained when the filter 1 is in contact with a liquid.

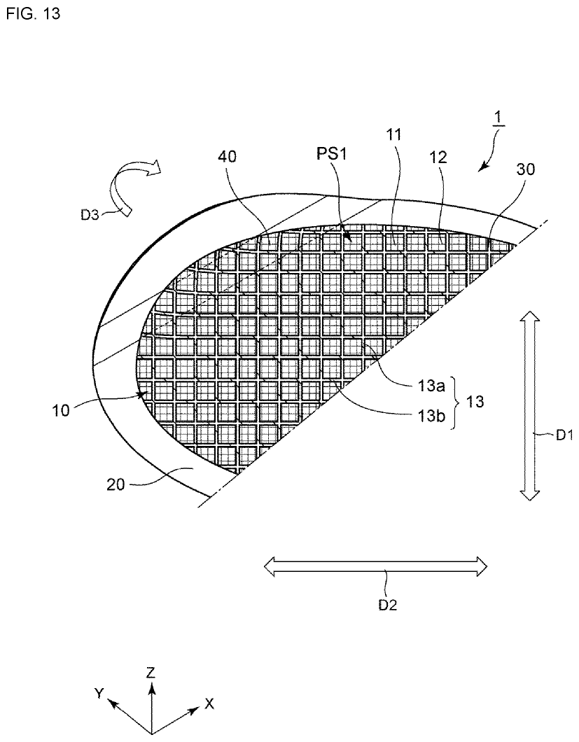

FIG. 13 is an enlarged perspective view of part of a curved portion 40. As illustrated in FIG. 13, the reinforcement 13 is located on the first main surface PS1 side of the filtering portion 10. The reinforcement 13 includes the plurality of first reinforcement members 13a extending in the first direction D1 and the plurality of second reinforcement members 13b extending in the second direction D2 intersecting the first direction D1. In Embodiment 1, the plurality of first reinforcement members 13a and the plurality of second reinforcement members 13b are orthogonal to one another on the XY plane.

The curved portion 40 is warped to the first main surface PS1 side in a third direction D3 intersecting the first direction D1 and the second direction D2 when viewed from the first main surface PS1 side of the filtering portion 10. Specifically, the curved portion 40 is curved in an arch shape in the third direction D3 that turns around the axis of the X direction, between the first direction D1 and the second direction D2 on the XY plane.

Since the first direction D1 and the second direction D2 are the extending directions of the plurality of first reinforcement members 13a and the plurality of second reinforcement members 13b, respectively, the mechanical strength is relatively high in these directions. However, in the third direction D3 different from the first direction D1 and the second direction D2, the curved portion 40 can be formed easily.

In Embodiment 1, the area occupied by the plurality of curved portions 40 is smaller than the area occupied by the flat portion 30 in the first main surface PS1 (see FIGS. 3 and 4). Note that the area occupied by the plurality of curved portions 40 is not limited to this configuration. The area occupied by the plurality of curved portions 40 may be larger than the area occupied by the flat portion 30. For example, in the first main surface PS1, the ratio of the area occupied by the plurality of curved portions 40 may be 1% to 100%. It is preferable that the ratio of the area occupied by the plurality of curved portions 40 in the first main surface PS1 be 5% to 50%. It is more preferably that the ratio of the area occupied by the plurality of curved portions 40 in the first main surface PS1 be 15% to 50%.

[Method of Manufacturing Filter]

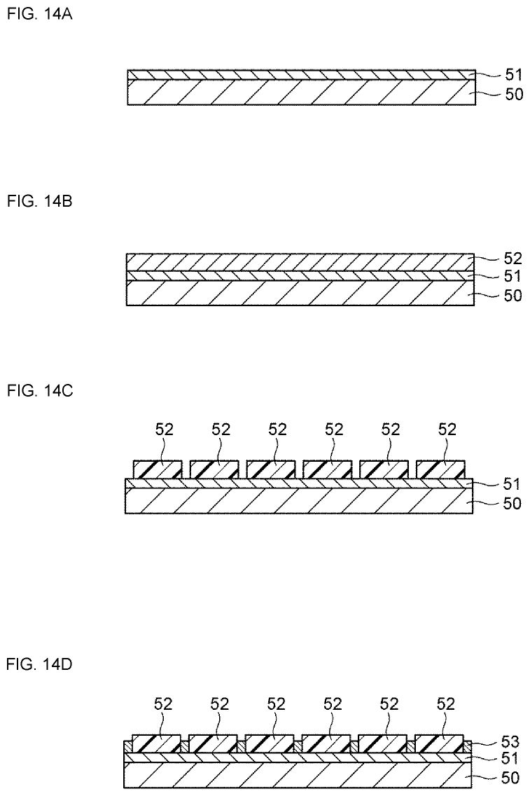

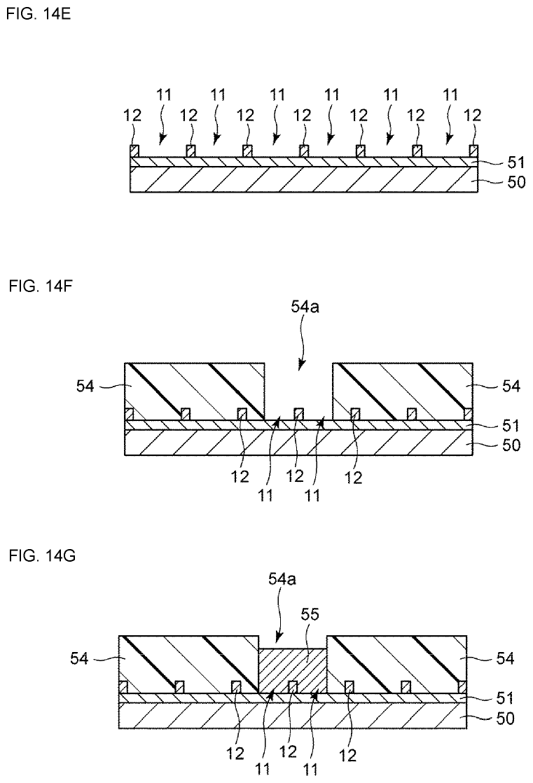

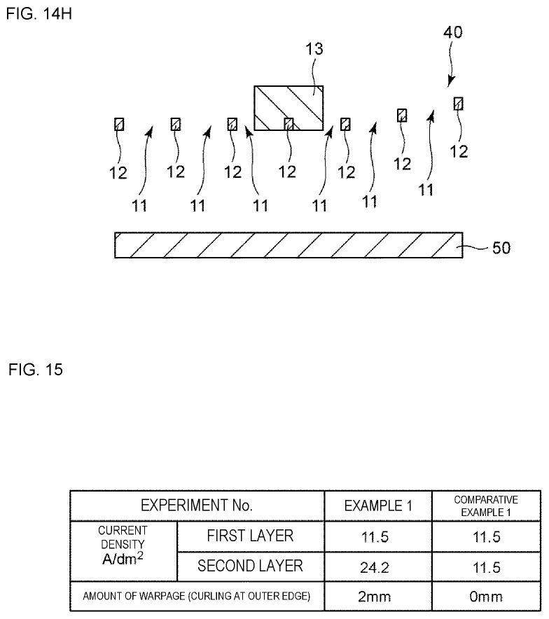

An example of a method of manufacturing the filter 1 will be described with reference to FIGS. 14A to 14H.

As illustrated in FIG. 14A, a Cu film 51 is formed on a substrate 50. For example, the Cu film 51 is formed by sputtering by using a sputtering-film formation device. Alternatively, the Cu film 51 may be formed by vapor deposition by using a vapor deposition device. In this process, to improve the adhesiveness between the substrate 50 and the Cu film 51, a Ti film may be formed between the substrate 50 and the Cu film 51.

As illustrated in FIG. 14B, a resist is applied onto the Cu film 51 and then dried to form a resist film 52. For example, a photosensitive positive liquid resist (Pfi-3A, available from Sumitomo Chemical Co., Ltd) is applied onto the Cu film 51 by using a spin coater. Then, the resist is heated and dried by using a hot plate to form the resist film 52.

As illustrated in FIG. 14C, the resist film 52 is exposed and developed to remove the portions of the resist film 52 corresponding to the filter base 12. For example, an i-line stepper (FPA-3030i5+, available from Canon Inc.) is used for the exposure device.

The development is performed by using a paddle developer. TMAH (Tetramethylammonium hydroxide) is used for the developer liquid. After exposure and development, washing with water and drying are performed.

As illustrated in FIG. 14D, electrolytic plating is performed by using an electrolytic plating device. This process forms a first layer 53, which is a plating film, at the portions where the resist film 52 has been removed.

As illustrated in FIG. 14E, the resist film 52 is stripped with a stripping solution NMP (N-methyl-2-pyrrolidone) by using a resist stripping device capable of a high-pressure spray process. After that, the first layer 53 is cleaned with IPA (isopropyl alcohol), washed with water, and dried. With these processes, the filter base 12 having a plurality of through holes 11 is formed.

As illustrated in FIG. 14F, a resist film 54 is formed on the filter base 12 except the portion 54a corresponding to the reinforcement 13 and the portion (not illustrated) corresponding to the frame 20. For example, a resist is applied to the filter base 12 and dried to form the resist film 54. The resist film 54 is exposed and developed to remove the portion 54a of the resist film 54 corresponding to the reinforcement 13 and the portion of the resist film 54 corresponding to the frame 20.

As illustrated in FIG. 14G, electrolytic plating is performed by using an electrolytic plating device. This process forms a second layer 55, which is a plating film, at the portion 54a corresponding to the reinforcement 13 and the portion corresponding to the frame 20, in other words, the portions where the resist film 54 is not formed. The current density in the electrolytic plating for the second layer 55 differs from that for the first layer 53.

As illustrated in FIG. 14H, the resist film 54 is stripped, and the Cu film 51 is removed by etching.

Through these processes, the filter 1 can be formed.

In the manufacturing method described above, the current density in the electrolytic plating for the second layer 55 forming the reinforcement 13 and the frame 20 differs from that for the first layer 53 forming the filter base 12. This makes it possible to form the curved portions 40.

FIG. 15 is a table illustrating the results of an experiment in which the amount of warpage was measured with current density taken as a parameter. As illustrated in FIG. 15, in Example 1, a filter was produced in a condition that the current densities for the first layer 53 and the second layer 55 were different, and the amount of warpage of the filter was measured. Specifically, in Example 1, the current density in the electrolytic plating for the first layer 53 was 11.5 A/dm², and the current density in the electrolytic plating for the second layer 55 was 24.2 A/dm². In Comparative Example 1, a filter was produced in a condition that the current densities for the first layer 53 and the second layer 55 were the same, and the amount of warpage of the filter was measured. Specifically, in Comparative Example 1, the current density in the electrolytic plating for the first layer 53 and the second layer 55 was 11.5 A/dm².

In Example 1, curved portions 40 were formed in the filter, and the amount of warpage was 2 mm. In contrast, in Comparative Example 1, curved portions 40 were not formed in the filter, and hence, the amount of warpage was 0 mm.

A higher current density causes greater internal stress (shrinkage rate) in the plating film. Hence, in the case in which the current densities for the first layer 53 and the second layer 55 are different, the internal stress (shrinkage rate) differs between the first layer 53 and the second layer 55. In the manufacturing method described above, the difference in the internal stress (shrinkage rate) is utilized to form the curved portions 40.

As described above, the filter 1 with the curved portions 40 can be manufactured by performing electrolytic plating in a condition that the current densities for forming the first layer 53 and the second layer 55 are different.

### Advantageous Effects

The filter 1 according to Embodiment 1 provides the following advantageous effects.

The filter 1 includes the first main surface PS1, the second main surface PS2 opposite to the first main surface PS1, and the filter base 12 having the plurality of through holes 11 causing the first main surface PS1 and the second main surface PS2 to communicate with each other. The filter base 12 includes the plurality of curved portions 40 warped to the first main surface PS1 side.

This configuration improves the usability of the filter 1. Specifically, since the filter 1 has the plurality of curved portions 40 warped to the first main surface PS1 side or the second main surface PS2, it is easy to distinguish between the front and back of the filter 1.

In addition, when the filter 1 is picked up with a tool such as tweezers, it is easy to pick up the filter by pinching a curved portion 40 with tweezers. For example, when removing the filter 1 from a holder after filtration, the user can easily pick up the filter 1 by pinching a curved portion 40 with tweezers. Thus, it is easy to remove the filter 1 from the holder. In addition, the filter 1 is less likely to break than a flat filter.

In addition, in a case in which a plurality of filters 1 are stacked for storage, the curved portions 40 make it easy to handle the filters 1.

The filter 1 further includes the frame 20 surrounding the periphery of the filter base 12 and extending along the outer peripheral shape of the filter base 12. This configuration also improves the mechanical strength of the filter 1. In addition, this configuration makes it easy to form the plurality of curved portions 40.

The filter 1 further includes the reinforcement 13 located on the filter base 12 and having a thickness t2 greater the thickness t1 of the filter base 12. This configuration also improves the mechanical strength of the filter 1. In addition, this configuration ensures the mechanical strength of the curved portions 40.

The reinforcement 13 includes the plurality of first reinforcement members 13a extending in the first direction D1 and the plurality of second reinforcement members 13b extending in the second direction D2 intersecting the first direction D1, when viewed from the first main surface PS1 side of the filter base 12. The plurality of curved portions 40 are warped to the first main surface PS1 side in the third direction D3 intersecting the first direction D1 and the second direction D2, when viewed from the first main surface PS1 side of the filter base 12. This configuration makes it possible to control the direction in which the curved portions 40 are warped. Specifically, since the plurality of first reinforcement members 13a and the plurality of second reinforcement members 13b extend in the first direction D1 and the second direction D2, respectively, the mechanical strength is relatively high in these directions. In the third direction D3 intersecting the first direction D1 and the second direction D2, the plurality of first reinforcement members 13a and the plurality of second reinforcement members 13b are not arranged, and accordingly, the filter base 12 is likely to be curved in the third direction D3. Since the filter 1 is relatively likely to be warped in the third direction D3, it is easy to form the curved portions 40.

The amount of warpage L1 of the plurality of curved portions 40 is 4×10⁻⁴ times the outer diameter d of the filter to 0.1 times the outer diameter d of the filter. This configuration further improves the usability of the filter 1. For example, when the filter 1 is removed from a holder, it is easy to insert the tips of tweezers on both sides of the curved portion 40.

The filter base 12 has the flat portion 30 in the center of the filter base 12 where the first main surface PS1 and the second main surface PS2 are flat. The plurality of curved portions 40 are formed with the flat portion 30 in between in cross-sectional view. This configuration further improves the usability of the filter 1. Also, the flat portion 30 ensures filtration performance.

In the first main surface PS1, the ratio of the area occupied by the plurality of curved portions 40 is 1% to 100%. This configuration further improves the usability of the filter 1.

The filter 1 contains at least one of a metal and a metal oxide as a main component. This configuration further improves the mechanical strength of the filter.

Although the description of Embodiment 1 is based on an example in which the filter 1 has a plurality of curved portions 40, the present disclosure is not limited to this configuration. For example, the filter 1 needs only to have one or more curved portions 40.

Although the description of Embodiment 1 is based on an example in which the curved portions 40 are warped to the first main surface PS1 side, the present disclosure is not limited to this configuration. For example, the curved portions 40 may be warped to the second main surface PS2 side. The direction in which the curved portions 40 are warped may be determined by, for example, adjusting the current densities in the electrolytic plating for forming the first layer 53 and the second layer 55, the dimensions of the reinforcement 13, the dimensions of the frame 20, and/or the like as appropriate.

Although the description of Embodiment 1 is based on an example in which the curved portions 40 are curved in an arch shape, the present disclosure is not limited to this configuration. For example, the curved portions 40 need only to be warped to the first main surface PS1 side or the second main surface PS2 side, and the shapes of the curved portions 40 are not limited to arch shapes.

Although the description of Embodiment 1 is based on an example in which the filter 1 includes the reinforcement 13 and the frame 20, the present disclosure is not limited to this configuration. The reinforcement 13 and the frame 20 are not essential constituents.

<Variations>

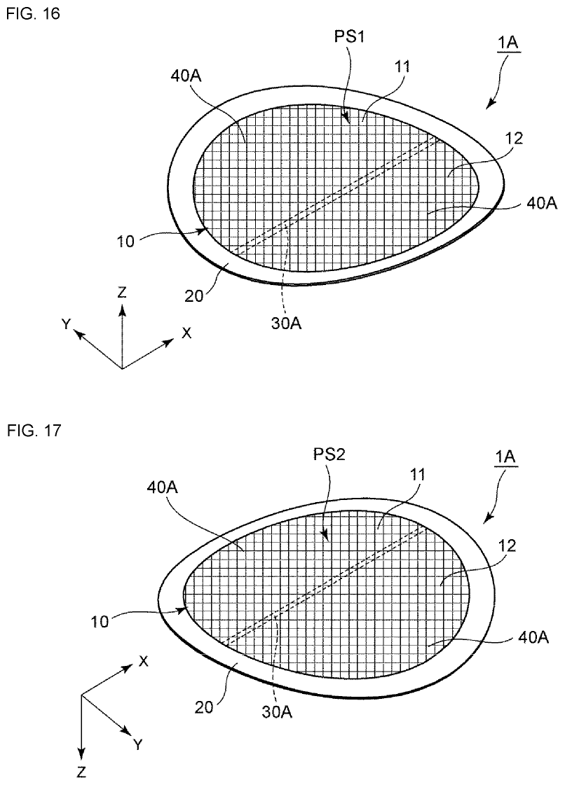

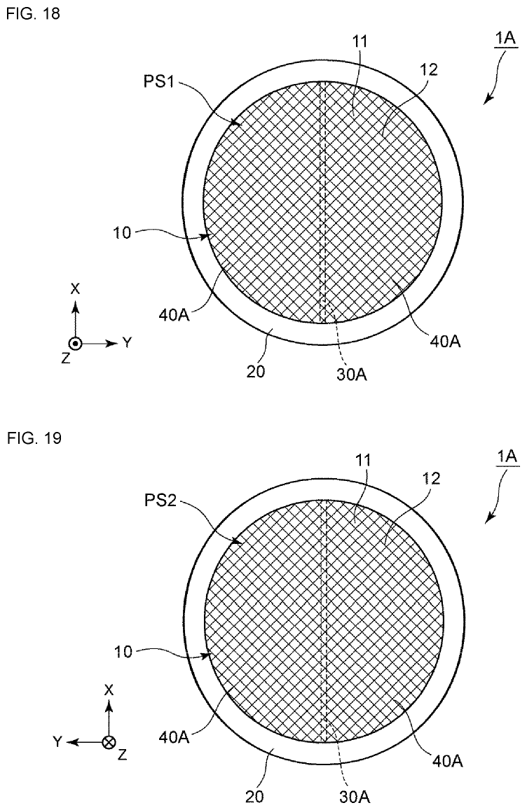

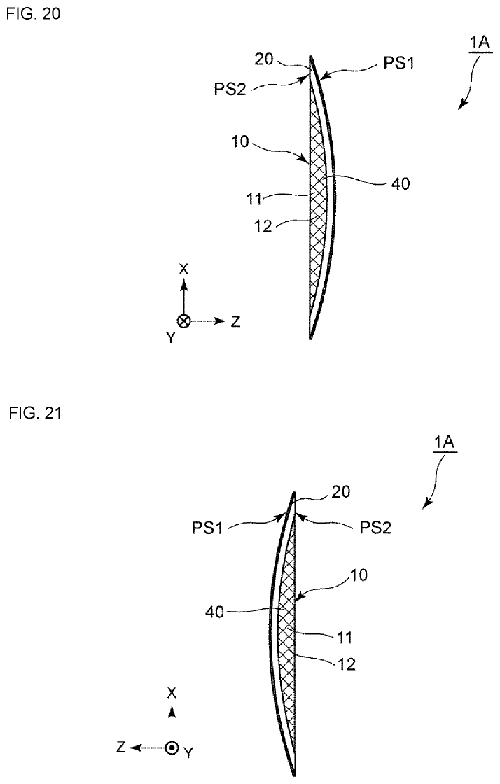

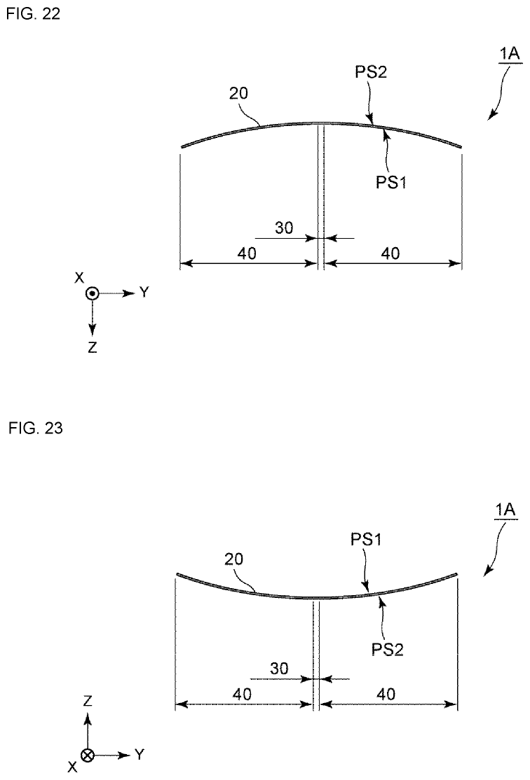

A filter 1A of a variation will be described with reference to FIGS. 16 to 23. FIG. 16 is a perspective view of the filter 1A of the variation from a first main surface PS1 side. FIG. 17 is a perspective view of the filter 1A of the variation from a second main surface PS2 side. FIG. 18 is a front view of the filter 1A of the variation. FIG. 19 is a rear view of the filter 1A of the variation. FIG. 20 is a left side view of the filter 1A of the variation. FIG. 21 is a right side view of the filter 1A of the variation. FIG. 22 is a plan view of the filter 1A of the variation. FIG. 23 is a bottom view of the filter 1A of the variation.

As illustrated in FIGS. 16 to 23, in the filter 1A, the ratio of the area occupied by curved portions 40A to the area occupied by a flat portion 30A in the first main surface PS1 is larger than in the filter 1 of Embodiment 1. Specifically, almost all of the filter 1A consists of the curved portions 40.

The configuration of the filter 1A also improves the usability as with the filter 1 of Embodiment 1. This configuration provides an advantageous effect that also when a plurality of filters 1A are stacked for storage, it is easy to stack and handle the plurality of filters 1A.

Although the description of the filter 1A of the variation is based on an example in which the filter 1A has a flat portion 30, the present disclosure is not limited to this configuration. For example, a configuration in which the filter 1A does not have a flat portion 30 is possible.

Although a preferred embodiment has been fully described with reference to the accompanying drawings, various changes and modifications are clearly possible for those skilled in the art. Notwithstanding, it should therefore be understood that the scope of the invention is to be defined by the claims.

Since the filter of the present disclosure has high usability, it is useful for filtration of a fluid containing a filtration target substance.

### REFERENCE SIGNS LIST

1 FILTER
10 FILTERING PORTION
11 THROUGH HOLE
12 FILTER BASE
12a FIRST BASE MEMBER
12b SECOND BASE MEMBER
13 REINFORCEMENT
13a FIRST REINFORCEMENT MEMBER
13b SECOND REINFORCEMENT MEMBER
20 FRAME
30 FLAT PORTION
40 CURVED PORTION
50 SUBSTRATE
51 Cu FILM
52 RESIST FILM
53 FIRST LAYER
54 RESIST FILM
55 SECOND LAYER
D1 FIRST DIRECTION
D2 SECOND DIRECTION
D3 THIRD DIRECTION
PS1 FIRST MAIN SURFACE
PS2 SECOND MAIN SURFACE

1. A filter comprising:
a filtering portion having a first main surface, a second main surface opposite to the first main surface, and a plurality of through holes connecting the first main surface and the second main surface with each other, wherein
the filtering portion includes one or more curved portions warped in a direction toward a side of the first main surface or a side of the second main surface.

2. The filter according to claim 1, further comprising a frame surrounding a periphery of the filtering portion and extending along an outer peripheral shape of the filtering portion.

3. The filter according to claim 1, further comprising a reinforcement on the filtering portion.

4. The filter according to claim 3, wherein a thickness of the reinforcement is greater than that of the filtering portion.

5. The filter according to claim 3, wherein
the reinforcement includes a plurality of first reinforcement members extending in a first direction and a plurality of second reinforcement members extending in a second direction intersecting the first direction when viewed from the side of the first main surface, and
the one or more curved portions are warped to the side of the first main surface or the side of the second main surface in a third direction intersecting the first direction and the second direction when viewed from the side of the first main surface.

6. The filter according to claim 5, wherein the plurality of first reinforcement members and the plurality of second reinforcement members are arranged at equal intervals.

7. The filter according to claim 1, wherein an amount of warpage of the one or more curved portions is 4×10⁻⁴ times an outer diameter of the filter to 0.1 times the outer diameter of the filter.

8. The filter according to claim 1, wherein an amount of warpage of the one or more curved portions is 4×10⁻³ times an outer diameter of the filter to 0.1 times the outer diameter of the filter.

9. The filter according to claim 1, wherein an amount of warpage of the one or more curved portions is 0.02 times an outer diameter of the filter to 0.1 times the outer diameter of the filter.

10. The filter according to claim 1, wherein
the filter base has a flat portion where the first main surface and the second main surface are flat, the flat portion being located in a center of the filtering portion, and
the one or more curved portions include first and second curved portions positioned such that the flat portion is located in between the first and second curved portions in a cross-sectional view of the filter.

11. The filter according to claim 10, wherein the first and second curved portions are located at a part of an outer periphery of the filter.

12. The filter according to claim 10, further comprising:
a frame surrounding a periphery of the filtering portion and extending along an outer peripheral shape of the filtering portion,
wherein the frame includes a part of the flat portion and parts of the first and second curved portions.

13. The filter according to claim 1, wherein a ratio of an area occupied by the one or more curved portions in the first main surface is 1% to 100%.

14. The filter according to claim 1, wherein a ratio of an area occupied by the one or more curved portions in the first main surface is 5% to 50%.

15. The filter according to claim 1, wherein a ratio of an area occupied by the one or more curved portions in the first main surface is 15% to 50%.

16. The filter according to claim 1, wherein the filter contains at least one of a metal and a metal oxide as a main component thereof.

17. The filter according to claim 1, wherein a thickness of the filtering portion is 0.5 μm to 20 μm.

18. The filter according to claim 1, wherein a thickness of the filtering portion is 1.0 μm to 3 μm.
